SEQUENCE LISTING (3 sequences)

SEQ ID NO: 1 - protein, 5 aa, Artificial Sequence
  Synthetic peptide
FFRWG

SEQ ID NO: 2 - protein, 5 aa, Artificial Sequence
  Synthetic peptide
FFRWX

SEQ ID NO: 3 - protein, 5 aa, Artificial Sequence
  Synthetic peptide
FFRWG

(19) United States
(12) Patent Application Publication
(10) Pub. No.: US 2008/0242600 A1
(43) Pub. Date: Oct. 2, 2008

(54) Backbone Cyclized Melanocortin Stimulating Hormone (Alpha Msh) Analogs

(76) Inventors: Chaim Gilon, Jerusalem (IL); Amnon Hoffman, Jerusalem (IL); Yaniv Linde, Jerusalem (IL); Shmuel Hess, Nes Ziona (IL)

Correspondence Address: WINSTON & STRAWN LLP;PATENT DEPARTMENT, 1700 K STREET, N.W., WASHINGTON, DC, 20006, US

(21) Appl. No.: 11/915,793

(22) PCT Filed: May 31, 2006
(86) PCT No.: PCT/IL2006/000640

§ 371 Date: May 20, 2008

Related U.S. Application Data
(60) Provisional application No. 60/685,482, filed on May 31, 2005.

(51) Int. Cl. A61K 38/12; C07K 5/12; A61P 3/04; A61P 3/10

(52) U.S. Cl. 514/11; 530/321

(57) ABSTRACT

Backbone cyclized peptides which are α-melanocortin stimulating hormone (αMSH) analogs, having improved Melanocortin-4 receptor agonist activity are disclosed. The backbone cyclized peptide analogs disclosed possess unique and superior properties over other analogs, such as metabolic stability, increased oral bioavailability, improved intestinal permeability and pharmacological activity in-vivo. Pharmaceutical compositions that include the backbone cyclized αMSH analogs, and methods of using such compositions for the treatment of metabolic disorders including obesity are also disclosed.

### FIELD OF THE INVENTION

The present invention relates to melanocortin stimulating hormone (αMSH) analogs, to pharmaceutical compositions containing same, and to methods for using such compounds for the treatment of metabolic disorders including obesity.

### BACKGROUND OF THE INVENTION

### Treatment of Obesity

The obesity rate worldwide is increasing and is currently considered as a core epidemic of the Western world in the twenty first century. More than 50% of the U.S. population is considered overweight, with >25% diagnosed as clinically obese. The statistical data show that obesity starts already at a young age—15% of the children and juveniles suffer from overweight, three fold higher than has been reported 25 years ago. Therefore, there is a clear economic and medical rationale to develop therapies that would prevent obesity. Many scientists and pharmaceutical companies all over the world are currently searching for suitable pharmacological solutions to tackle this problem.

Upper body obesity is the strongest risk factor known for diabetes mellitus type 2, and is a strong risk factor for cardiovascular disease. Obesity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia (see, e.g., Kopelman, Nature 404: 635-43, 2000). It reduces life span and carries a serious risk of co-morbidities as described above, as well as disorders such as infections, varicose veins, acanthosis nigricans, eczema, exercise intolerance, insulin resistance, hypertension hypercholesterolemia, cholelithiasis, orthopedic injury, and thromboembolic disease (Rissanen et al., BMJ 301: 835-7, 1990). Obesity is also a risk factor for the group of conditions called insulin resistance syndrome, or “Syndrome X”.

Obesity is derived from chronic disequilibrium between the amount of calories, which enters the body, and the energy that has been utilized and wasted at the same time. Thus, eating high calories food and limited physical activity lead to fatness.

Energy stores are maintained relatively constant in mammals, in spite of a large variation in food availability and physical activity. This tight regulation is achieved by an endocrine feedback loop initiated by leptin. Leptin, produced by adipocytes, signals the nutritional status to the hypothalamus. Its concentration in plasma is correlated with adipose tissue mass and decreases during fasting. Leptin signal triggers a neuroendocrine response involving neuropeptides that modulate appetite and energy expenditure. Some of them also influence pituitary secretions, thus mediating the adaptive hormonal response associated with food deprivation: changes in circulating thyroid hormone levels, suppression of reproductive capacity and linear growth. Orexigenic peptides (neuropeptide Y, oxerins, etc.) are suppressed by leptin whereas anorexigenic signals are stimulated.

Currently, all the available medications for the treatment of obesity are suboptimal. Currently available treatments of obesity include orlistat and sibutramine.

Orlistat (tetrahydrolipstatin) is a synthetic drug derived from a naturally occurring lipase inhibitor produced by Streptomyces molds. It binds covalently to the active site of pancreatic lipase, the principal enzyme responsible for hydrolyzing triglyceride, which accounts for 99% of dietary fat; it also inhibits other gut and extra-intestinal lipases but its action is restricted to the gut lumen because it is essentially nonabsorbable. Orlistat at therapeutic doses (120 mg three times daily) blocks the digestion and absorption of about 30% of dietary fat, and this accounts for part but not all of its weight-reducing effect; the rest may be due to the patient choosing to avoid the high-fat foods which can provoke gastrointestinal side-effects.

Sibutramine is a centrally acting appetite suppressant that also has mild thermogenic properties. It acts by enhancing the action of two monoamines that act in the hypothalamus and other brain regions to induce negative energy deficits, namely serotonin (5-HT) and noradrenaline. When injected centrally in rodents and lower primates, both 5-HT and noradrenaline inhibit feeding and increase energy expenditure by stimulating the sympathetic outflow to the thermogenic tissues. Sibutramine blocks the reuptake of both monoamines, and therefore increases their availability in the synaptic cleft; unlike the fenfluramines, sibutramine does not stimulate the release of 5-HT from serotonergic nerve terminals. The inhibition of noradrenaline re-uptake increases sympathetic tone, consequences of which include both the desirable thermogenic effect and the undesirable cardiovascular side effects of rise in blood pressure and pulse rate. Because of its actions on both monoamines, sibutramine is referred to as an ‘SNRI’ (serotonin/noradrenaline reuptake inhibitor).

Potential CNS targets for novel anti-obesity drugs include various peptides, which are involved in food uptake and energy regulation. These peptides are the subject of intense research for conversion into orally active anti-obesity drugs. These include: Neuropeptide Y (NPY), Orexins and Melanocortins. It should be emphasized that these peptide analogs do not cross the intestinal wall thus do not have orally bioavailability.

### Melanocortin Agonist Peptides

One of the proposed solutions for the pharmacotherapy of this significant health problem is to regulate the biochemical pathways, which control food consumption and metabolic balance in the body.

The “melanocortin pathway” is a key endocrine regulating system of energy balance (Cummings and Schwartz 2000, Nat. Genet. 26(1):8-9). The state of art of the pharmacological approach to control caloric intake is focused on the late stages of the “melanocortin pathway” feedback cascade process. This process includes binding of the catabolic endogenic neuropeptide melanocortin stimulating hormone (αMSH) to its melanocortin subtype 4 (MC4) receptor, and produces an agonistic effect. This subtype of melanocortin (MC) receptor regulates the rate in which the fats are burned and thus affect the weight homeostasis (Luevano, C. H., et al., Biochemistry, 2001. 40: p. 6164-6179). The MC4 receptor, due to its direct involvement in feeding behavior, is a target for the design of selective potent agonist therapeutics to treat obesity and the design of selective antagonists to treat anorexia.

The central melanocortin system plays a pivotal role in regulation of energy homeostasis. The melanocortin peptides (α, β, γ-melanocyte stimulating hormones and adrenocorticotropin hormone ACTH) are the endogenous agonist ligands for the melanocortin receptors and are derived by post-translational processing of the pro-opiomelanocortin (POMC) gene transcript.

All of the melanocortin peptide agonists contain the core tetrapeptide His-Phe-Arg-Trp that has been attributed to the ligand selectivity and stimulation of the melanocortin receptors. Thus, the tetra peptide His-Phe-Arg-Trp can be used as a lead for designing therapeutic agents against obesity (Haskell-Luevano, Lim et al. 2000, Peptides. 21(1):49-57).

The melanocortin family contains five receptors (MC1R-MC5R) identified to date, which stimulate the cAMP second messenger signal transduction pathway.

The sequence homology between the melanocortin family members-ranges from 35 to 60% (Cone, et al., Rec. Prog. Hormone Res. 1996, 51: 287-318), but these receptors differ in their functions. For example, the MC1-R is a G-protein coupled receptor that regulates pigmentation in response to the αMSH, which is a potent agonist of MC1-R. Agonism of the MC1-R receptor results in stimulation of the melanocytes, which causes eumelanin and increases the risk for cancer of the skin. Agonism of MC1-R can also have neurological effects. Stimulation of MC2-R activity can result in carcinoma of adrenal tissue. The effects of agonism of the MC3-R and MC5-R are not yet known. All of the melanocortin receptors respond to the peptide hormone class of melanocyte stimulating hormones (MSH). Because of their different functions, simultaneous agonism of the activities of multiple melanocortin receptors has the potential of causing unwanted side effects. Therefore, it is desirable to obtain receptor-selective agonists.

Oral drug administration remains the most preferred route of systemic administration for chemical entities particularly for the treatment of chronic diseases such as obesity. However, as a result of extensive intestinal metabolic degradation and poor intestinal permeability, peptides suffer from poor oral bioavailability. Enzymatic stability of peptides in the gut lumen and the brush border is a major factor dominating peptide oral bioavailability, as proteolytic enzymes are abundant at these regions. Hence, proteolytic enzymes considerably lessen the ability of intact peptides to reach the systemic circulation following oral administration. For tetra (and larger) peptides, more than 90% of the proteolytic activity is by enzymes bounded to the brush border membrane. The poor permeability of peptides is usually due to a combination of incompatible physicochemical properties, resulting in low cellular penetration. Successful oral delivery of peptides will depend therefore, on strategies designed to alter the physicochemical characteristics of these potential drugs in order to improve both metabolic stability and intestinal permeability without affecting their pharmacological activity.

The peptide analogs of the endogenous αMSH have poor metabolic stability both in the blood and in the gastrointestinal (GI) tract (ultra-short half life) and therefore, cannot be used as therapeutic compounds against obesity.

It has been demonstrated that, when injected into the third ventricle of the brain or intraperitoneally, a cyclic heptapeptide analog of αMSH having MC4-R agonist activity caused long lasting inhibition of food intake in mice. This effect was reversible when co-administered with a MC4-R antagonist (Fan, et al., Nature, 1997 385: 165-168). Therefore, agonists of MC4-R activity would be useful in treating or preventing obesity.

U.S. Patent Application Publication No. 20010056179 discloses selective linear peptides with melanocortin-4 receptor (MC4-R) agonist activity. WO 2003/095474 discloses specific peptide derivatives having melanocortin-4 receptor agonist activity. WO 2005/009950 discloses piperidine derivatives which are selective agonists of the human melanocortin-4 receptor.

U.S. Patent Application Publication No. 20020143141 discloses selective lactam-bridged cyclic peptides with MC4-R agonist activity. WO 02/18437 discloses peptides cyclized via disulfide or lactam bridges having MC4-R agonist activity useful for treatment of obesity. WO 2003/006604 discloses cyclic peptides as potent and selective melanocortin-4 receptor agonists. WO 2005/030797 discloses cyclic peptides comprising 7-12 amino acid residues having MC4-R agonist activity. However, these peptide analogs do not cross the intestinal wall thus do not have orally bioavailability.

### Improved Peptide Analogs

As a result of major advances in organic chemistry and in molecular biology, many bioactive peptides can now be prepared in quantities sufficient for pharmacological and clinical use. Thus in the last few years new methods have been established for the treatment and diagnosis of illnesses in which peptides have been implicated.

However, the use of peptides as therapeutic and diagnostic agents is limited by the following factors: a) low tissue penetration; b) low metabolic stability towards proteolysis in the gastrointestinal tract and in serum; c) poor absorption after oral ingestion, in particular due to their relatively high molecular mass or the lack of specific transport systems or both; d) rapid excretion through the liver and kidneys; and e) undesired side effects in non-target organ systems, since peptide receptors can be widely distributed in an organism.

It would be desirable to achieve peptide analogs with greater specificity thereby achieving enhanced clinical selectivity. It would be most beneficial to produce conformationally constrained peptide analogs overcoming the drawbacks of the native peptide molecules, thereby providing improved therapeutic properties.

A novel conceptual approach to the conformational constraint of peptides was introduced by Gilon, et al., (Biopolymers, 1991, 31, 745) who proposed backbone cyclization of peptides. Backbone cyclization is a general method by which conformational constraint is imposed on peptides. In backbone cyclization, atoms in the peptide backbone (N and/or C) are interconnected covalently to form a ring.

The theoretical advantages of this strategy include the ability to effect cyclization via the carbons or nitrogens of the peptide backbone without interfering with side chains that may be crucial for interaction with the specific receptor of a given peptide. Further disclosures by Gilon and coworkers (WO 95/33765, WO 97/09344, U.S. Pat. No. 5,723,575, U.S. Pat. No. 5,811,392, U.S. Pat. No. 5,883,293, U.S. Pat. No. 6,265,375 and U.S. Pat. No. 6,407,059), provided methods for producing building units required in the synthesis of backbone cyclized peptide analogs. The successful use of these methods to produce backbone cyclized peptide analogs of bradykinin analogs (U.S. Pat. No. 5,874,529), and backbone cyclized peptide analogs having somatostatin activity (WO 98/04583, WO 99/65508, U.S. Pat. No. 5,770,687, U.S. Pat. No. 6,051,554 and U.S. Pat. No. 6,355,613) was also disclosed.

There remains a need for synthetic orally bioavailable αMSH peptidomimetic analogs having increased in vivo stability, to be used for the treatment of metabolic disorders, e.g., obesity. It would be desirable to achieve αMSH peptide analogs with greater affinity and selectivity to the MC4 receptor, thereby achieving pharmaceutical compounds for the treatment of metabolic disorders.

### SUMMARY OF THE INVENTION

The present invention provides therapeutically useful αMSH analogs that are backbone cyclic peptide analogs, pharmaceutical compositions comprising these αMSH analogs and methods of use thereof. In particular the present invention provides receptor specific αMSH backbone cyclized analogs useful for the treatment of metabolic disorders. The novel analogs according to the present invention having agonist activity to Melanocortin-4 receptor (MC-4R) associated with obesity may be used in the treatment of metabolic disorders including obesity. The analogs provided according to the present invention have prolonged metabolic stability, high intestinal permeability, oral availability and pharmacological activity in-vivo.

According to one aspect of the present invention, backbone cyclized αMSH analogs are provided, comprising a peptide sequence of four to twelve amino acids that incorporates at least one building unit, the building unit containing one nitrogen atom of the peptide backbone connected to a bridging group comprising a disulfide, amide, thioether, thioester, imine, ether, or alkene bridge, wherein at least said one building unit is connected via the bridging group to a moiety selected from the group consisting of a second building unit, a side chain of an amino acid residue of the peptide sequence, and a N-terminal amino acid residue, to form a cyclic structure. Preferably, the peptide sequence incorporates five to eight amino acids.

According to some embodiments, the bridging group is a chemical linker having the general Formula (VII):

Z-(CH₂)ₘ-M-(CH₂)ₙ Formula (VII)

wherein m and n are each independently an integer for 1 to 8; M is selected from the group consisting of a disulfide, amide, thioether, thioester, imine, ether, or alkene bridge and Z is absent or is a molecule comprising two carboxylic groups.

One embodiment of the present invention, is a backbone cyclic peptide analog of the general Formula I (SEQ ID NO: 2):

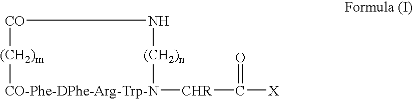

wherein R is the side chain of an amino acid, X is OH, NH₂ or an ester, m denotes an integer from 1 to 8 and n denotes an integer from 1 to 8.

According to some embodiments, m denotes an integer from 2 to 5 and n denotes an integer from 2 to 6.

Another embodiment according to the present invention is a backbone cyclic peptide analog of Formula II (SEQ ID NO: 3):

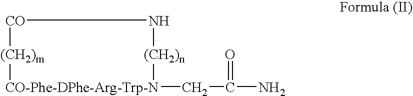

wherein m denotes an integer from 1 to 8 and n denotes an integer from 1 to 8.

According to some embodiments, m denotes an integer from 2 to 5 and n denotes an integer from 2 to 6.

Preferred peptides according to Formula II are those in which ring size is from about 20 to about 27 atoms. More preferred peptides are selected from the group consisting of:

a peptide according to Formula II wherein n=2, m=2;
a peptide according to Formula II wherein n=3, m=3;
a peptide according to Formula II wherein n=3, m=2;
a peptide according to Formula II wherein n=3, m=5;
a peptide according to Formula II wherein n=2, m=4.

One currently preferred embodiment is a peptide of Formula II wherein n=2 and m=2 denoted herein BBC-1.

A further embodiment according to the present invention is a backbone cyclic peptide analog of Formula III:

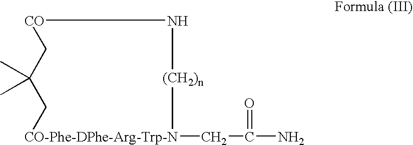

wherein n denotes an integer from 1 to 8.

According to some embodiments, n denotes an integer from 2 to 6.

Preferred peptides according to Formula III are selected from the group consisting of:

a peptide according to Formula III wherein n=2;
a peptide according to Formula III wherein n=3;
a peptide according to Formula III wherein n=4;
a peptide according to Formula III wherein n=6.

Another embodiment according to the present invention is a backbone cyclic peptide analog of Formula IV:

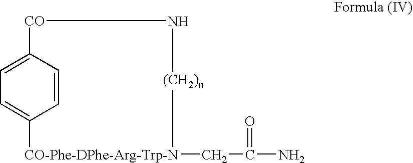

wherein n denotes an integer from 1 to 8.

According to some embodiments, n denotes an integer from 2 to 6.

Preferred peptides according to Formula IV are selected from the group consisting of:

a peptide according to Formula IV wherein n=2;
a peptide according to Formula IV wherein n=3;
a peptide according to Formula IV wherein n=4;
a peptide according to Formula IV wherein n=6.

According to another aspect the present invention provides pharmaceutical compositions comprising as an active ingredient a backbone cyclic peptide analog of αMSH. According to one embodiment, the pharmaceutical composition is formulated for oral administration.

According to a further aspect, the present invention provides a method for treatment or prophylaxis of diseases or disorders which are associated with melanocortin-4-receptor activity, comprises administering to a subject in need thereof a therapeutically effective amount of a pharmaceutical composition comprising as an active ingredient a backbone cyclic peptide analog of αMSH.

According to some embodiments, the disorders are metabolic disorders. According to one embodiment, the metabolic disorder is diabetes. According to a preferred embodiment, the metabolic disorder is obesity.

According to another embodiment, the amount of the active ingredient is in the range of from about 10 to 1000 μg/kg.

According to a still another aspect, the present invention provides the use of a backbone cyclic peptide analog of αMSH for the preparation of a medicament for the treatment or prevention of diseases or disorders which are associated with melanocortin-4-receptor activity.

These and other embodiments of the present invention will become apparent in conjunction with the figures, description and claims that follow.

### BRIEF DESCRIPTION OF THE FIGURES

FIG. 1 depicts a scheme for the synthesis of a library of peptides according to the invention (m=2, 3, 4, 5; n=2, 3, 4, 6).

FIG. 2 describes the synthesis of protected Glycine-derived building units.

FIG. 3 describes the general structures of the backbone cyclized (BBC) libraries, according to the invention.

FIG. 4 shows the Permeability coefficient values (Papp) of MC-4 peptides from library I compared to standard molecules with known intestinal permeability: mannitol indicates low permeability while testosterone and propranolol represent high intestinal permeability.

FIG. 5 demonstrates the effect of backbone cyclization of peptides on metabolic stability in rat intestinal brush border membranes.

FIG. 6 shows the chemical structure of backbone cyclic peptide BBC-1.

FIG. 7 demonstrates the effect of BBC-1 on food consumption in mice. Data are expressed as the mean ±SEM. Statistical analysis made by one-way ANOVA with Dunnett post testing:*, P<0.05.

FIGS. 8 A-B show characterization of BBC1 as performed by reversed phase HPLC (RP-HPLC) (8A) and MALDI-TOF MS (8B).

### DETAILED DESCRIPTION OF THE INVENTION

It is now disclosed that the backbone cyclic peptidomimetic approach has led to the discovery of backbone cyclic peptide αMSH analogs having agonist activity to Melanocortin-4 receptor. The αMSH analogs are useful in the treatment of metabolic disorders including obesity, preferably by oral administration.

According to the present invention, backbone cyclic peptide analogs of αMSH which possess high intestinal permeability, prolonged metabolic stability, oral availability and pharmacological activity in-vivo were selected from libraries of backbone cyclized peptide analogs.

As used herein the term “backbone cyclic peptide analog” refers to a sequence of amino acid residues wherein at least one nitrogen or carbon of the peptide backbone is joined to a moiety selected from another such nitrogen or carbon, to a side chain or to one of the termini of the peptide. Furthermore, one or more of the peptide bonds of the sequence may be reduced or substituted by a non-peptidic linkage.

The term “amino acid” refers to compounds, which have an amino group and a carboxylic acid group, preferably in a 1,2- 1,3-, or 1,4-substitution pattern on a carbon backbone. α-Amino acids are most preferred, and include the 20 natural amino acids (which are L-amino acids except for glycine) which are found in proteins, the corresponding D-amino acids, the corresponding N-methyl amino acids, side chain modified amino acids, the biosynthetically available amino acids which are not found in proteins (e.g., 4-hydroxy-proline, 5-hydroxy-lysine, citrulline, ornithine, canavanine, djenkolic acid, β-cyanolanine), and synthetically derived α-amino acids, such as amino-isobutyric acid, norleucine, norvaline, homocysteine and homoserine. β-Alanine and 7-amino butyric acid are examples of 1,3 and 1,4-amino acids, respectively, and many others are well known to the art. Statine-like isosteres (a dipeptide comprising two amino acids wherein the CONH linkage is replaced by a CHOH), hydroxyethylene isosteres (a dipeptide comprising two amino acids wherein the CONH linkage is replaced by a CHOHCH₂), reduced amide isosteres (a dipeptide comprising two amino acids wherein the CONH linkage is replaced by a CH₂NH linkage) and thioamide isosteres (a dipeptide comprising two amino acids wherein the CONH linkage is replaced by a CSNH linkage) are also useful residues for this invention.

The amino acids used in this invention are those, which are available commercially or are available by routine synthetic methods. Certain residues may require special methods for incorporation into the peptide, and sequential, divergent or convergent synthetic approaches to the peptide sequence are useful in this invention. Natural coded amino acids and their derivatives are represented by three-letter codes according to IUPAC conventions. When there is no indication, the L isomer was used. The D isomers are indicated by “D” before the residue abbreviation.

Conservative substitutions of amino acids as known to those skilled in the art are within the scope of the present invention. Conservative amino acid substitutions includes replacement of one amino acid with another having the same type of functional group or side chain e.g. aliphatic, aromatic, positively charged, negatively charged. These substitutions may enhance oral bioavailability, penetration into the central nervous system, targeting to specific cell populations and the like. One of skill will recognize that individual substitutions, deletions or additions to peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

### 1) Alanine (A), Serine (S), Threonine (T);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V); and

### 6) Phenylalanine (F), Tyrosine (Y), Tryptophan (W).

As used herein “peptide” indicates a sequence of amino acids linked by peptide bonds. The peptides according to the present invention comprise a sequence of 4 to 12 amino acid residues, preferably 5 to 8 residues. A peptide analog according to the present invention may optionally comprise at least one bond, which is an amide-replacement bond such as urea bond, carbamate bond, sulfonamide bond, hydrazine bond, or any other covalent bond.

Salts and esters of the peptides of the invention are encompassed within the scope of the invention. Salts of the peptides of the invention are physiologically acceptable organic and inorganic salts. Functional derivatives of the peptides of the invention covers derivatives which may be prepared from the functional groups which occur as side chains on the residues or the N- or C-terminal groups, by means known in the art, and are included in the invention as long as they remain pharmaceutically acceptable, i.e., they do not destroy the activity of the peptide and do not confer toxic properties on compositions containing it. These derivatives may, for example, include aliphatic esters of the carboxyl groups, amides of the carboxyl groups produced by reaction with ammonia or with primary or secondary amines, N-acyl derivatives of free amino groups of the amino acid residues formed by reaction with acyl moieties (e.g., alkanoyl or carbocyclic aroyl groups) or O-acyl derivatives of free hydroxyl group (for example that of seryl or threonyl residues) formed by reaction with acyl moieties.

The term “analog” indicates a molecule, which has the amino acid sequence according to the invention except for one or more amino acid changes. The design of appropriate “analogs” may be computer assisted. A peptide analog according to the present invention may optionally comprise at least one bond which is an amide-replacement bond such as urea bond, carbamate bond, sulfonamide bond, hydrazine bond, or any other covalent bond.

The term “peptidomimetic” means that a peptide according to the invention is modified in such a way that it includes at least one non-coded residue or non-peptidic bond. Such modifications include, e.g., alkylation and more specific methylation of one or more residues, insertion of or replacement of natural amino acid by non-natural amino acids, replacement of an amide bond with other covalent bond. A peptidomimetic according to the present invention may optionally comprises at least one bond which is an amide-replacement bond such as urea bond, carbamate bond, sulfonamide bond, hydrazine bond, or any other covalent bond. The design of appropriate “peptidomimetic” may be computer assisted.

By “stable compound” or “stable structure” is meant herein a compound that is sufficiently robust to survive isolation to a useful degree of purity from a reaction mixture, and formulation into an efficacious therapeutic agent.

The term, “substituted” as used herein, means that any one or more hydrogen atoms on the designated atom is replaced with a selection from the indicated group, provided that the designated atom's normal valency is not exceeded, and that the substitution results in a stable compound.

When any variable (for example n, m, etc.) occurs more than one time in any constituent or in any formula herein, its definition on each occurrence is independent of its definition at every other occurrence. Also, combinations of substituents and/or variables are permissible only if such combinations result in stable compounds.

The term “receptor agonist” refers to a molecule that can combine with a receptor on a cell to produce a physiologic reaction typical of a naturally occurring substance.

The term “agonist of MC-4 receptor” preferably means that the molecules are capable of mimicking at least one of the actions of αMSH mediated through the MC receptor subtype 4.

As used herein, the phrase “therapeutically effective amount” means that amount of novel backbone cyclized peptide analog or composition comprising same to administer to a host to achieve the desired results for the indication disclosed herein, such as but not limited to obesity.

### Backbone Cyclization of Peptides

Backbone cyclized analogs are peptide analogs cyclized via bridging groups attached to the alpha nitrogens or alpha carbonyl of amino acids that permit novel non-peptidic linkages. In general, the procedures utilized to construct such peptide analogs from their building units rely on the known principles of peptide synthesis; most conveniently, the procedures can be performed according to the known principles of solid phase peptide synthesis. During solid phase synthesis of a backbone cyclized peptide the protected building unit is coupled to the N-terminus of the peptide chain or to the peptide resin in a similar procedure to the coupling of other amino acids. After completion of the peptide assembly, the protective group is removed from the building unit's functional group and the cyclization is accomplished by coupling the building unit's functional group and a second functional group selected from a second building unit, a side chain of an amino acid residue of the peptide sequence, and a N-terminal amino acid residue.

As used herein the term “backbone cyclic peptide” or “backbone cyclic analog” denote an analog of a linear peptide which comprising a peptide sequence of preferably 3 to 24 amino acids that incorporates at least one building unit, said building unit containing one nitrogen atom of the peptide backbone connected to a bridging group comprising an amide, thioether, thioester, disulfide, urea, carbamate, or sulfonamide, wherein at least one building unit is connected via said bridging group to form a cyclic structure with a moiety selected from the group consisting of a second building unit, the side chain of an amino acid residue of the sequence or a terminal amino acid residue.

A “building unit” (BU) indicates an N^{α} or C^{α} derivatized amino acid. An N^{α} derivatized amino acid is represented by the general formula (V):

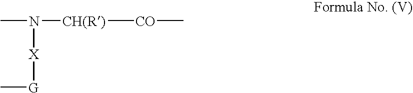

wherein X is a spacer group selected from the group consisting of allkylene, substituted alkylene, arylene, cycloalkylene and substituted cycloalkylene; R′ is an amino acid side chain, optionally bound with a specific protecting group; and G is a functional group selected from the group consisting of amines, thiols, alcohols, carboxylic acids, sulfonates, esters, and alkyl halides; which is incorporated into the peptide sequence and subsequently selectively cyclized via the functional group G with one of the side chains of the amino acids in said peptide sequence, with one of the peptide terminals, or with another co-functionalized amino acid derivative.

The present invention is exemplified by using N^{α} derivatized Glycine of the general formula (VI):

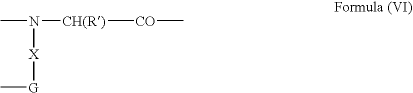

wherein X is alkylene, R′ is a hydrogen; and G is amine; which is incorporated into the peptide sequence and subsequently selectively cyclized via the functional group G with a carboxylic group attached to the N-terminus of said peptide sequence.

The building units in the present invention are depicted in their chemical structure as part of the peptide sequence or are abbreviated by the three letter code of the corresponding modified amino acid preceded by the type of reactive group (N for amine, C for carboxyl). For example, N-Gly describes a modified Gly residue with an amine reactive group thus, according to the present invention, N-Gly within a sequence of a backbone cyclized peptide is equal to NH—(CH₂)ₙ—N—CH₂—CONH₂

The methodology for producing the building units is described in international patent applications published as WO 95/33765 and WO 98/04583 and in U.S. Pat. Nos. 5,770,687 and 5,883,293 all of which are expressly incorporated herein by reference thereto as if set forth herein in their entirety.

The term “bridging group” according to the present invention refers to a chemical linker or spacer connecting a nitrogen atom of the peptide backbone to a second building unit, to a side chain of an amino acid residue of the sequence or to a terminal amino acid residue. According to some embodiments the chemical linker or spacer group is presented by the general Formula (VII):

Z-(CH₂)ₘ-M-(CH₂)ₙ Formula (VII)

wherein m and n are each independently an integer for 1 to 8; M is selected from the group consisting of a disulfide, amide, thioether, thioester, imine, ether, or alkene bridge and Z is absent or is a molecule comprising two carboxylic groups, such as a dicarboxylic acid residue. Non-limiting examples of Z according to the present invention are succinic acid residue and phthalic acid residue. Backbone cyclized peptides according to the present invention may be synthesized using any method known in the art, including peptidomimetic methodologies. These methods include solid phase as well as solution phase synthesis methods. Non-limiting examples for these methods are described hereby. Other methods known in the art to prepare compounds like those of the present invention can be used and are comprised in the scope of the present invention.

The methods for design and synthesis of backbone cyclized analogs according to the present invention are disclosed in U.S. Pat. Nos. 5,811,392; 5,874,529; 5,883,293; 6,051,554; 6,117,974; 6,265,375, 6,355,613, 6,407,059, 6,512,092 and international applications WO 95/33765; WO 97/09344; WO 98/04583; WO 99/31121; WO 99/65508; WO 00/02898; WO 00/65467 and WO 02/062819. All of these methods are incorporated herein in their entirety, by reference.

The most striking advantages of backbone cyclization are: 1) cyclization of the peptide sequence is achieved without compromising any of the side chains of the peptide thereby decreasing the chances of sacrificing functional groups essential for biological recognition (e.g. binding to specific receptors), and function; 2) optimization of the peptide conformation is achieved by allowing permutation of the bridge length, and bond type (e.g., amide, disulfide, thioether, thioester, urea, carbamate, or sulfonamide, etc.), bond direction, and bond position in the ring; 3) when applied to cyclization of linear peptides of known activity, the bridge can be designed in such a way as to minimize interaction with the active region of the peptide and its cognate receptor. This decreases the chances of the cyclization arm interfering with recognition and function.

The principles of the “backbone cyclic peptidomimetic” approach are based on the following steps: (i) elucidation of the active residues in the target protein (ii) design and modeling of an ensemble of prototypic backbone cyclic peptides that encompass the active residues and their conformation resemble that of the parent protein (iii) cycloscan of each backbone cyclic prototype until a lead compound is discovered (iv) structural analysis of the best lead and (v) optimization through iteration.

“Cycloscan” is a selection method based on conformationally constrained backbone cyclic peptide libraries that allows rapid detection of the most active backbone cyclic peptide derived from a given sequence as disclosed in WO 97/09344. The teachings of this disclosure are incorporated herein in their entirety by way of reference. The diversity of cycloscan, which includes modes of backbone cyclization, ring position, ring size and ring chemistry allows the generation of a large number of sequentially biased peptides that differ solely by their conformation in a gradual discrete manner.

### Pharmacology

Apart from other considerations, the fact that the novel active ingredients of the invention are peptides, peptide analogs or peptidomimetics, dictates that the formulation be suitable for delivery of these types of compounds. Although in general peptides are less suitable for oral administration due to susceptibility to digestion by gastric acids or intestinal enzymes. According to the present invention, novel methods of backbone cyclization are being used, in order to synthesize metabolically stable and oral bioavailable peptidomimetic analogs. The preferred route of administration of peptides of the invention is oral administration.

Other routes of administration are intra-articular, intravenous, intramuscular, subcutaneous, intradermal, or intrathecal.

Pharmaceutical compositions of the present invention may be manufactured by processes well known in the art, e.g., by means of conventional mixing, dissolving, granulating, grinding, pulverizing, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants for example polyethylene glycol are generally known in the art.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

For buccal administration, the compositions may take the form of tablets or lozenges formulated in conventional manner.

For administration by inhalation, the variants for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the peptide and a suitable powder base such as lactose or starch.

Pharmaceutical compositions for parenteral administration include aqueous solutions of the active ingredients in water-soluble form. Additionally, suspensions of the active compounds may be prepared as appropriate oily injection suspensions. Suitable natural or synthetic carriers are well known in the art (Pillai et al., Curr. Opin. Chem. Biol. 5, 447, 2001). Optionally, the suspension may also contain suitable stabilizers or agents, which increase the solubility of the compounds, to allow for the preparation of highly concentrated solutions. Alternatively, the active ingredient may be in powder form for reconstitution with a suitable vehicle, e.g., sterile, pyrogen-free water, before use.

The compounds of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of a compound effective to prevent, alleviate or ameliorate symptoms of a disease of the subject being treated. Determination of a therapeutically effective amount is well within the capability of those skilled in the art.

Toxicity and therapeutic efficacy of the peptides described herein can be determined by standard pharmaceutical procedures in cell cultures or experimental animals, e.g., by determining the IC50 (the concentration which provides 50% inhibition) and the LD50 (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition (e.g. Fingl, et al., 1975, in “The Pharmacological Basis of Therapeutics”, Ch. 1 p. 1).

The preferred doses for administration of such pharmaceutical compositions range from about 0.1 μg/kg to about 20 mg/kg body weight/day. Preferably, the amount of the active ingredient is in the range of from about 10 to 1000 μg/kg.

Depending on the severity and responsiveness of the condition to be treated, dosing can also be a single administration of a slow release composition, with course of treatment lasting from several days to several weeks or until cure is effected or diminution of the disease state is achieved. The amount of a composition to be administered will, of course, be dependent on the subject being treated, the severity of the affliction, the manner of administration, the judgment of the prescribing physician, and all other relevant factors.

### General Screening of αMSH Analogs

The αMSH analogs are typically tested in vitro for their inhibition of the natural peptide (Agouti Related Protein) binding to its melanocortin-4 (MC4) receptor. The analogs can be further tested in vitro for their influence on cyclic adenosine monophosphate (cAMP) levels. Intestinal permeability and metabolic stability of the analogs can be tested in vivo. The analogs can be further tested in vivo in preclinical models in order to identify the optimal mode of administration and proper dose, and to verify the safety and the efficacy of these new potential therapeutic drugs.

### Preferred Modes for Carrying Out the Invention

According to the present invention, novel peptide analogs, which are characterized in that they incorporate novel building units with bridging groups attached to the alpha nitrogens of alpha amino acids, are disclosed. Specifically, these compounds are backbone cyclized αMSH analogs comprising a peptide sequence of four to twelve amino acids, that incorporates at least one building unit, said building unit, containing one nitrogen atom of the peptide backbone connected to a bridging group comprising a disulfide, amide, thioether, thioester, imine, ether, or alkene bridge, wherein at least one building unit is connected via the bridging group to a second building unit, a side chain of an amino acid residue of the peptide sequence, or a N-terminal amino acid residue to form a cyclic structure. Preferably, the peptide sequence incorporates 4 to 12 residues, more preferably 5 to 8 amino acids.

According to the principles of the present invention backbone cyclic peptides based on the active region of the hormone αMSH that activate the MC4 receptor are provided. For this purpose libraries of backbone cyclic peptides based on the MC4R active parent sequence: Phe-D-Phe-Arg-Trp-Gly-NH₂ (SEQ ID NO: 1) were synthesized. All the peptides in the libraries have the parent sequence. They differ from each other by their ring size and ring chemistry.

All peptides were studied for MC4R functionality and selectivity as well as in-vitro intestinal absorption and intestinal metabolic degradation. One peptide, herein designated BBC-1 was found to be highly functional and selective while possessing high intestinal metabolic stability and permeability. In-vivo studies in mice showed reduced food consumption over a period of 24 hr of ˜40% when administrated orally.

A currently preferred embodiment according to the present invention is a backbone cyclic peptide analog of Formula II (SEQ ID NO: 3).

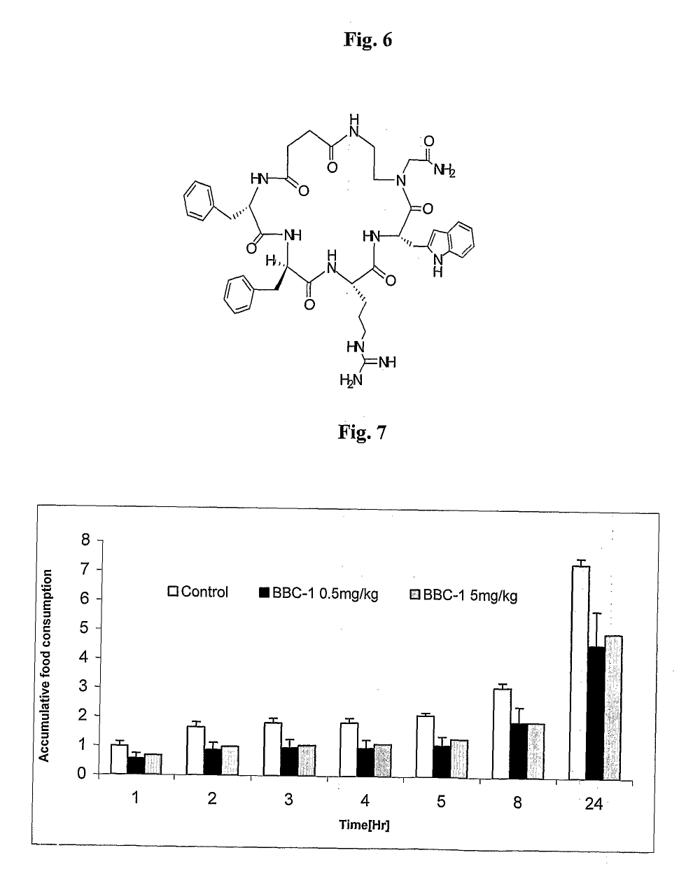

A currently preferred peptide of the invention is denoted herein BBC-1 (FIG. 6). BBC-1, chosen for its specific activation of MC4R was found to be enzymatically stable with enhanced in vitro intestinal permeability. Single oral administration of BBC-1 in mice resulted in decreased food consumption for 24 hours.

Backbone cyclic analogs of the present invention bind with high affinity to MC4 receptor. This receptor selectivity indicates the potential physiological selectivity in vivo. Furthermore, the present invention provides for the first time the possibility to obtain a panel of backbone cyclized analogs with specific MC4 receptor selectivity. This enables therapeutic uses in metabolic disorders including obesity.

The αMSH analogs of the present invention can be used for treating obesity or preventing overweight, regulating the appetite, inducing satiety, preventing weight regain after successful weight loss, increasing energy expenditure and treating a disease or state related to overweight or obesity.

The pharmaceutical compositions containing the αMSH analogs of the present invention may be formulated, at strength effective for administration by various means to a human or animal patient experiencing undesirably elevated body weight, either alone or as part of an adverse medical condition or disease, such as type II diabetes mellitus.

The αMSH analogs of the invention are useful as primary agents for the treatment of type II diabetes mellitus, and for the treatment of type I diabetes mellitus. The αMSH analogs according to the present invention are also useful as adjunctive agents for the treatment of type I, or type II diabetes.

The αMSH analogs can be used as therapies for diseases caused by, or coincident with aberrant glucose metabolism. The αMSH analogs can be used for delaying the progression from impaired glucose tolerance (IGT) to type II diabetes, and delaying the progression from type II diabetes to insulin requiring diabetes.

Having now generally described the invention, the same will be more readily understood through reference to the following examples, which are provided by way of illustration and are not intended to be limiting of the present invention.

### EXAMPLES

### Materials and Methods

### Peptide Synthesis

Protected amino acids, 9-fluorenylmethyloxycarbonyl-N-hydroxysuccinimide (Fmoc-OSu), bromo-tris-pyrrolidone-phosphonium hexafluorophosphate (PyBrop), Rink amide methylbenzhydrylamine (MBHA) polystyrene resins and many organic and supports for solid phase peptide synthesis (SPPS) were purchased from Nova Biochemicals (Laufelfingen, Switzerland). Bis(trichloromethyl)carbonate (BTC) was purchased from Lancaster (Lancashire, England), Trifluoroacetic acid (TFA) and solvents for high performance liquid chromatography (HPLC) were purchased from Bio-Lab (Jerusalem, Israel). Glyoxylic acid, 1,2-diaminoethane, 1,3-diaminopropane and 1,4-diaminobutane were purchased from Merck (Darmstadt, Germany), tetrakis (triphenylphosphine) palladium (0) was purchased from ACROS (Geel, Belgium).

Solvents for organic chemistry were purchased from Frutarom (Haifa, Israel). Nuclear magnetic resonance (NMR) spectra were recorded on a Bruker AMX-300 MHz spectrometer. Mass spectra were performed on a Finnigan LCQ DUO ion trap mass spectrometer. Thin layer chromatography (TLC) was performed on Merck F245 60 silica gel plates (Darmstadt, Germany). HPLC analysis was performed using a Vydac analytical RP column (C18, 4.6× 250 mm, catalog number 201TP54), and were carried out on a Merck-Hitachi L-7100 pump and a Merck-Hitachi L-7400 variable wavelength detector operating at 215 nm. The mobile phase consisted of a gradient system, with solvent A corresponding to water with 0.1% TFA and solvent B corresponding to acetonitrile (ACN) with 0.1% TFA. The mobile phase started with 95% A from 0 to 5 min followed by linear gradient from 5% B to 95% B from 5 to 55 min. The gradient remained at 95% B for an additional 5 min, and then was dropped to 95% A and 5% B from 60 to 65 min. The gradient remained at 95% A for additional 5 min to achieve column equilibration. The flow rate of the mobile phase was 1 mL/min. Peptide purification was performed by reversed phase HPLC (RP-HPLC) (on L-6200A pump, Merck-Hitachi, Japan), using a Vydac preparative RP column (C8, 22× 250 mm, catalog number 218TP1022). All preparative HPLC were carried out using a gradient system with solvent A corresponding to water with 0.1% TFA and solvent B corresponding to ACN with 0.1% TFA.

### Example 1

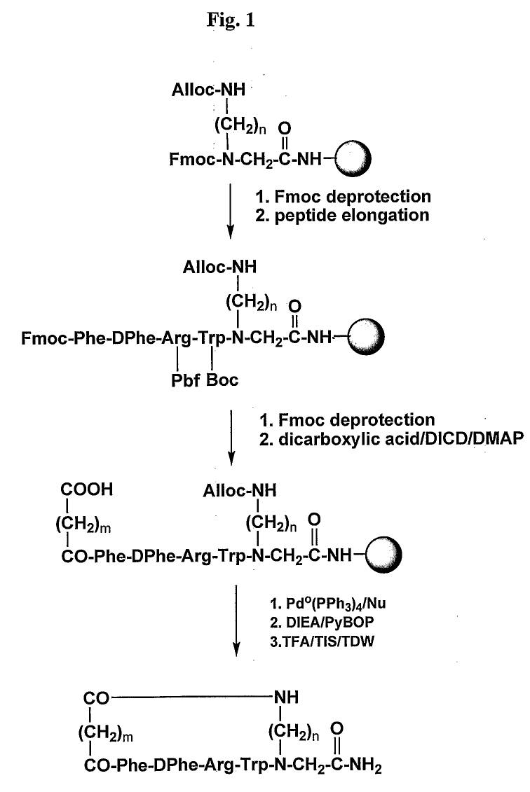

### Solid Phase Peptide Synthesis of the Backbone Cyclic αMSH Analogs (FIG. 1)

The synthesis was performed in a reaction vessel equipped with a sintered glass bottom, following general Fmoc chemistry protocols: Rink amide methylbenzhydrilamine (MBHA) resin (1 g, 0.66 mmol/g) was pre-swollen in N-methylpyrrolidone (NMP) for 2 h. Fmoc deprotection step was carried out with 20% piperidine in NMP (2×30 min), followed by washing with NMP (5×2 min) and DCM (2×2 min). Couplings of the building unit Fmoc-N^{α}(Ethylamine-Alloc)Gly-OH (Fmoc-GlyN2) to the resin and of Fmoc-amino-acid-OH (Fmoc-Axx-OH) to the building unit were carried out as follows: Fmoc-GlyN2 (3 eq., 1.98 mmol) and bis-(trichloromethyl) carbonate (BTC, triphosgene) (1 eq., 0.66 mmol) were suspended in DCM. 2,4,6-collidine (10 eq., 6.6 mmol) was added to the pre-cooled suspension in an ice bath. After all the solids were dissolved (about 1 min), the solution was poured onto the resin and shaken for 3 h at room temperature. This coupling cycle was repeated once more. At the end of the second coupling cycle, the peptidyl-resin was washed with DCM (5×2 min). Capping was carried out after the first amino acid and was repeated twice by reaction of the peptidyl-resin with a mixture of acetic anhydride (1.1 mL, 0.5 M), diisopropyl ethyl amine (DIEA) (0.5 mL, 0.125 M) in dimethyl formamide (DMF) (25 mL). Capping was followed with resin wash with DMF (5×2 min), DCM (2×2 min), and NMP (2×2 min). Coupling of Fmoc-Trp(BOC)—OH, Fmoc-Arg(Pbf)-OH, Fmoc-D-Phe-OH and Fmoc-Phe-OH were carried out using BTC as the coupling agent in the same way that was described above. The last amino acid on the peptidyl-resin (Phe) was acylated with 10 eq. of succinic anhydride (m=2), in NMP, for 2 h at room temperature, in the presence of 1 eq. DMAP and 10 eq. of DIEA.

The resin was washed with NMP (2×5 min) and DCM (2×5 min), dried overnight in a desiccator and removal of the Alloc protecting group from the building unit was performed with tetrakis(triphenylphosphine)Pd(0) (0.1 eq., 0.066 mmol) in NMP containing acetic acid (5%) and N-methyl morpholin (2.5%) under Argon. This step was carried out for 4 h with vigorous shaking in the dark. Washing steps were carried out with chloroform (8×2 min), and NMP with 0.5% DIEA (3×2 min). Following Alloc deprotection the peptide was cyclized by the addition of 6 eq. PyBoP and 12 eq. DIEA in NMP (repeated twice). Washing steps were carried out with NMP (5×2 min) and DCM (5×2 min). The peptidyl-resin was dried under vacuo over night.

Cleavage from the resin and removal of side chain protecting groups was carried out simultaneously using a pre-cooled mixture of 95% TFA, 2.5% TDW and 2.5% triisopropylsilane (TIS). After the resin was added, the mixture was agitated for 30 min in an ice bath, and then was shaken for 2.5 h at room temperature. The combined TFA filtrates were evaporated to dryness by a stream of nitrogen. The oily residue was triturated three times with cold ether to remove the scavengers, and the ether was removed by centrifugation. The dry crude peptide was dissolved in ACN/H₂O (1:1) and lyophilized.

### Example 2

### Synthesis of the Building Units

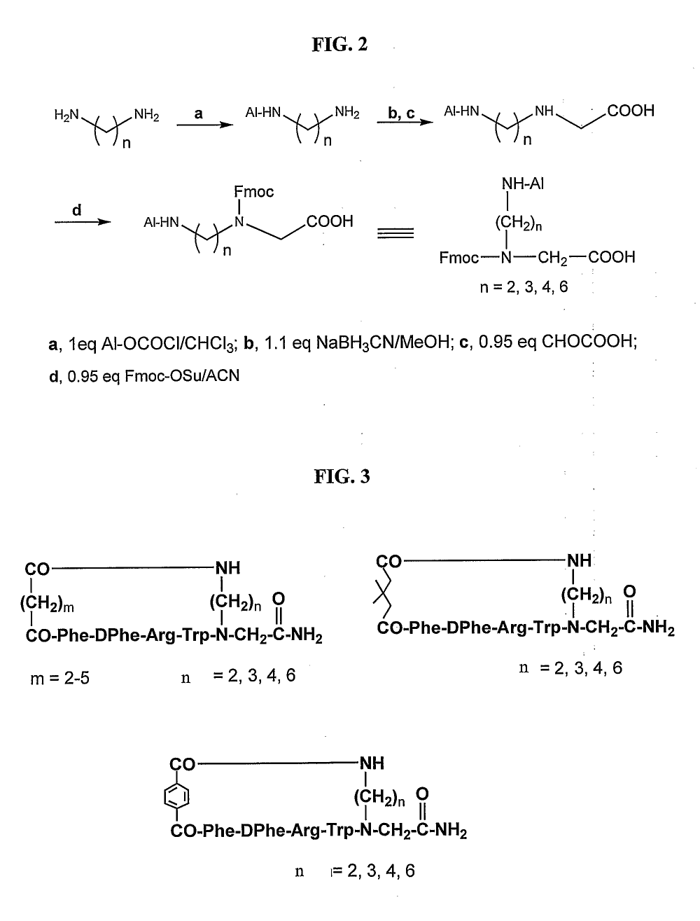

### (i) Synthesis of Glycine-Derived Building Unit was Performed According to FIG. 2

### (ii) Preparation of Alloc-NH(CH₂)₂-4NH₂ (1)

1 mol of 1,2-Diaminoethane, 1,3-Diaminopropane or 1,4-Diaminobutane (10 eq., 66.85 m″l, 82.40 m″l or 98.05 m″l, respectively) was dissolved in Chloroform (500 mL) and cooled in an ice bath. To the cooled solution, 0.1 mol Allyl chloroformat (1 eq.) in Chloroform (250 mL) were added at 0° C. drop wise over 3 h and then stirred overnight at room temperature. The reaction mixture was washed with water (200 mL×2), dried over sodium sulfate and evaporated in vacuo.

### (iii) Synthesis of Alloc-NH—(CH₂), —NH—CH₂—COOH (2)

NaCNBH₃ (1.1 eq., 0.052 mol) was added in MeOH (100 mL). Compound (I) (0.0454 mol) was dissolved in MeOH (50 mL) and added to the NaCNBH3 solution. Glyoxilic acid (0.95 eq., 0.0434 mol) was added and the reaction was stirred over night. The MeOH was evaporated under reduced pressure.

### (iv) Synthesis of Fmoc-Gly (Nn)Alloc-OH (3)

The residue was dissolved in water (110 mL), and triethyl amine (11 mL, 0.079 mol) was added. Fmoc-OSu (9.82 g, 0.0291 mol) in AcCN (170 mL) was added, and the reaction was stirred for 4 h whereas the pH was kept alkaline with triethyl amine. The reaction mixture was washed with petroleum ether PE (180 mL×3) and ether:PE 7:3 (180 mL×3). The aqueous layer was acidified under cooling to pH_{—}3-4 with 2M HCl (10 mL), and extracted with ethyl acetate (EA) (150 mL×4). The organic layer was washed with 1M HCl (100 mL×2) and sat. KHSO₄ (100 mL×2), dried over Na2SO₄ and evaporated in vacuo to yield: 5.50 g, 0.0115 mol (39.5%) of colorless oil that was later solidified. The product was used for SPPS without further purification.

### Example 3

### Peptide Synthesis

The structures of the backbone cyclic peptides as well as their MS and purity are shown in Table 1. All the peptides have the same sequence namely Phe-DPhe-Arg-Trp-Gly-NH₂ as well as the same lactam ring position: between the N^{α} of Gly and the amino terminus. The peptides in the library differ from each other by their ring size and ring chemistry. The ring size ranges from 20 atoms (peptide MCR4-1) to 25 atoms (peptide MC4-14). The differences in the ring chemistry is achieved by changing the relative size of the alkyl chains n and m that leads to peptides with the same ring size, but with different position of the amide bond in the lactam ring. For example, peptides MCR4-6, MCR4-10 and MCR4-11 all have a ring size of 22 atoms but they differ from each other by n and m. Thus peptide MCR4-6 has n=3 and m=3 whereas peptide MCR4-10 has n=2 and m=4 and peptide MCR4-11 has n=4 and m=2.

### Example 4

### Evaluation of Intestinal Permeability

Growth and maintenance of cells; Caco-2 cells are obtained from ATCC and then grown in 75 cm² flasks with approximately 0.5-10⁶ cells/flask at 37° C. in 5% CO₂ atmosphere and at relative humidity of 95%. The culture growth medium consisted of Dulbecco's Modified Eagle Medium (DMEM) supplemented with 10% heat-inactivated fetal bovine serum (FBS), 1% nonessential amino acids (NEAA), and 2 mM L-glutamine. The medium is replaced twice weekly.

Preparation of cells for transport studies; for the transport studies cells in a passage range of 60-66 are seeded at density of 25×10⁵ cells/cm² on untreated culture inserts of polycarbonate membrane with 0.4 μm pores and surface area of 1 cm². The culture inserts containing Caco-2 monolayer are placed in 24 transwells plates 12 mm, Costar™. The culture medium is changed every other day. Transport studies are performed 21-23 days after seeding, when the cells were fully differentiated and the TEER values are stable (300-500 μcm²).

Experiment, protocol: Transport study is initiated by medium removal from both sides of the monolayer and replacement with apical buffer (550 μl) and basolateral buffer (1200 μl), both warmed to 37° C. The cells are incubated for 30 minutes period at 37° C. with shaking (100 cycles/min). After incubation period the buffers are removed and replaced with 1200 μl basolateral buffer at the basolateral side. Test solutions are warmed previously to 37° C. and added (600 μl) to the apical side of the monolayer. 50 μl samples are taken from the apical side immediately at the beginning of the experiment, resulting in 550 μl apical volume during the experiment. For the period of the experiment the cells are kept at 37° C. with shaking. At predicted times (30, 60, 90, 120, 150 and 180 min.), the 200 μl samples are taken from the basolateral side and replaced with the same volume of flesh basolateral buffer to maintain a constant volume.

### Example 5

### Assessment of Intestinal Metabolic Stability

Brush-border membrane vesicles (BBMVs) were prepared from combined duodenum, jejunum, and upper ileum by a Ca++precipitation method (PEERCE). The intestines of 5 male Wistar rats, 200-250 g, were rinsed with ice cold 0.9% Nacl and freed of mucos, the mucosa was scraped off the luminal surface with glass slides and put immediately into buffer containing 50 nM Kcl and 10 mM Tris-HCl (pH 7.5, 4° C.) and the mixture homogenated by Ploytron (Polytron PT 1200, Kinematica AG, Switzerland). CaCl was added to a final concentration of 10 mM. The homogenate was left shaking for 30 min at 4° C. and afterwards centrifuged at 10,000 g for 10 min (centrifuge) the supernatant was then centrifuged at 48,000 g for 30 min an additional two purification steps were undertaken by suspending the pellet in 300 mM mannitol and 10 mM Hepes/Tris (pH 7.5) and centrifuge 24,000 g/hr. Purification of brush border membranes was assayed using the brush border membrane enzyme markers GGT, LAP and alkaline phosphatase. During the course of these studies, enrichment in brush border membrane enzymes varied between 13- and 18-fold.

### Example 6

### Receptor Binding Assays

Transfected CHO cells are washed with binding buffer 8 and distributed into 96-well plates (approximately 40,000 cells/well). The cells are then incubated for 2 h at 37° C. with 0.05 ml binding buffer in each well, containing a constant concentration of [¹²⁵I] NDP-α-MSH and appropriate concentrations of an unlabelled ligand. After incubation, the cells are washed with 0.2 ml of ice-cold binding buffer and detached from the plates with 0.2 ml of 0.1 N NaOH. Radioactivity is counted (Wallac, Wizard automatic gamma counter) and data analyzed with a software package for radioligand binding analyses (Wan System, Umea, Sweden) by fitting it to formulas derived from the law of mass-action by the method generally referred to as computer modeling. The binding assays are performed in duplicate wells.

### Example 7

### Determination of Receptors Activation

### cAMP Assay as a Probe

cAMP Accumulation Assays: 48 h after transfection, CHO cells are washed once with PBS and then detached from the plate with PBS containing 0.02% EDTA (Sigma). The detached cells are harvested by centrifugation and resuspended in Hanks' balanced salt solution (Invitrogen) containing 0.5 mM IBMX, 2 mM HEPES, pH 7.5 (IBMX buffer). After incubation at 37° C. for 15 min to allow for IBMX uptake, 0.4 ml of cell suspension (5×10⁵ cells/ml) is added to 0.1 ml of IBMX buffer containing various concentrations of agonists or 10 μM forskolin. The cells are subsequently incubated at 37° C. for 15 min to allow for cAMP accumulation. The activity is terminated by adding 0.5 ml of 5% trichloroacetic acid, and cAMP released from lysed cells is assayed by the cAMP ¹²⁵I scintillation proximity assay system (Amersham Biosciences).

EC₅₀ values are calculated with a 95% confidence interval using GraphPad Prism software (using nonlinear regression analysis fitted with a sigmoidal dose-response curve with variable slope).

### Example 8

### Characterization of Three Backbone Cyclic Peptide Libraries

TABLE 1
Characterization of Library 1 (Formula II)
Library 1

| | Characterization | | | | | |
|---|---|---|---|---|---|---|
| | | | | | Mw + H | |
| Peptide | | | Size | Mw-Cal | Found | Purity |
| name | n | m | ring | (gr/mol) | (gr/mol) | (%) |
| BBC1 | 2 | 2 | 20 | 836.42 | 837.30 | 84.3 |
| BBC4 | 2 | 3 | 21 | 850.43 | 851.01 | 82.2 |
| BBC6 | 3 | 3 | 22 | 864.45 | 865.13 | 84.2 |
| BBC7 | 3 | 5 | 24 | 892.48 | 893.40 | 89.3 |
| BBC8 | 3 | 2 | 21 | 850.43 | 851.60 | 88.8 |
| BBC9 | 2 | 5 | 23 | 878.46 | 879.47 | 97.6 |
| BBC10 | 2 | 4 | 22 | 864.45 | 865.70 | 97.8 |
| BBC11 | 4 | 2 | 22 | 864.45 | | IP |
| BBC12 | 4 | 3 | 23 | 878.46 | 879.33 | 97.8 |
| BBC13 | 4 | 4 | 24 | 892.48 | 894.00 | 79.2 |
| BBC14 | 4 | 5 | 25 | 906.49 | | IP |
| BBC15 | 3 | 4 | 23 | 878.46 | 879.72 | 96.2 |
| BBC22 | 6 | 2 | 24 | 892.48 | | IP |
| BBC23 | 6 | 3 | 25 | 906.49 | | IP |
| BBC24 | 6 | 4 | 26 | 920.52 | | IP |
| BBC25 | 6 | 5 | 27 | 948.58 | | IP |

Three backbone cyclic peptide libraries based on the active region of the hormone αMSH that activates the MC4 receptor (see FIG. 3) were synthesized and characterized, (see Tables 1-3). The Backbone cyclic peptides from library I were tested for their intestinal permeability in comparison to known standards. As shown in FIG. 4, the peptide BBC1 (FIG. 6) possesses high intestinal permeability.

The IC₅₀ values of these peptides on the MC4R are shown in Table 4. All the peptides have similar IC₅₀ values to the natural hormone (70 nM), with two analogs having better affinity.

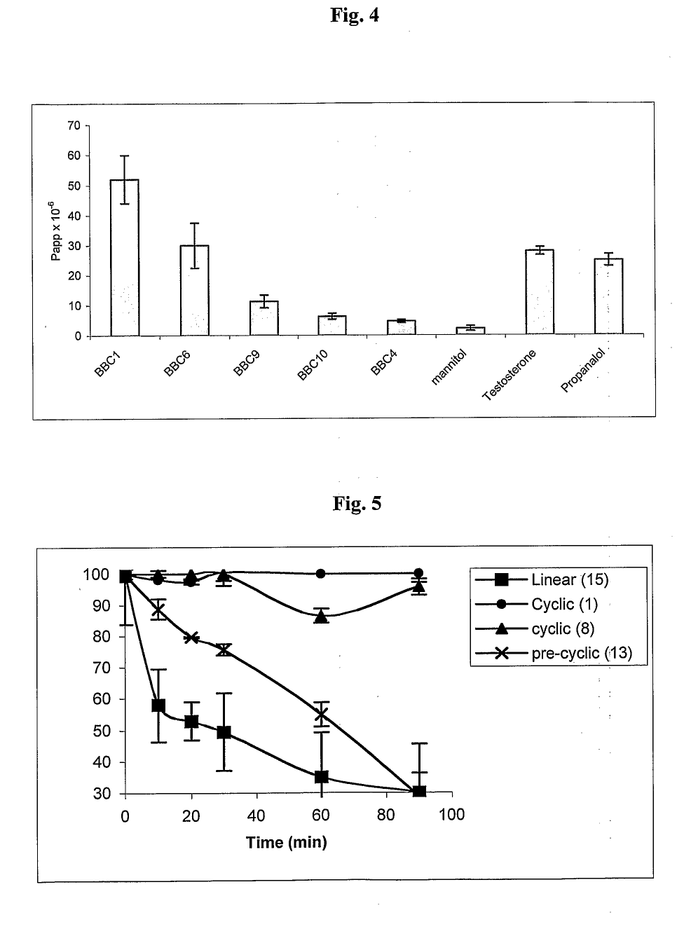

The intestinal metabolic stability of the peptides is shown in FIG. 5. The cyclic peptides have prolonged metabolic stability as compared to the linear analogs.

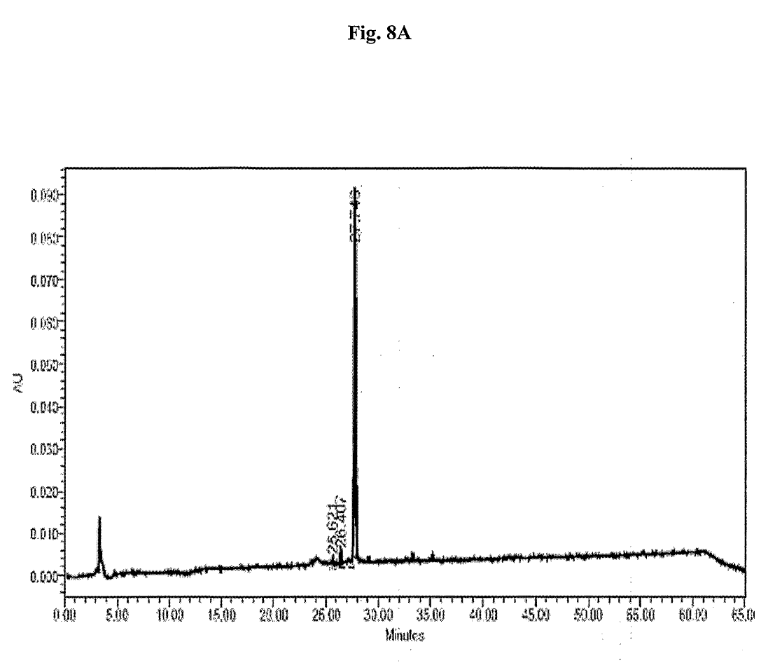

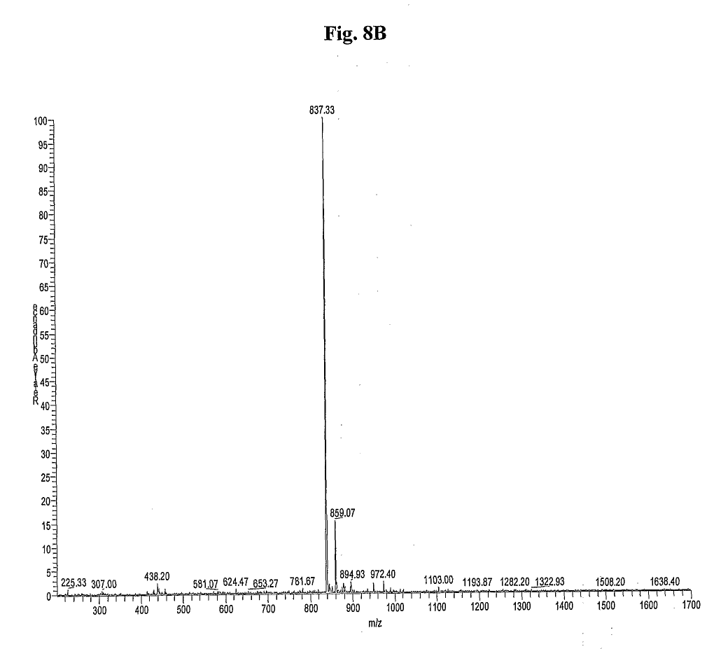

Characterization of the BBC1 peptide was performed by reversed phase HPLC (RP-HPLC) and matrix-associated laser desorption ionization time-of-flight mass spectroscopy (MALDI-TOF MS) (FIGS. 8 A and B respectively).

TABLE 2
Characterization of Library 2 (Formula III)
Library 2

Characterization

| | | | Mw + H | |
|---|---|---|---|---|
| Peptide | | Mw − Cal | Found | Purity |
| name | n | (gr/mol) | (gr/mol) | (%) |
| BBC17 | 2 | 878.00 | 879.79 | 95.3 |
| BBC19 | 3 | 892.06 | | N.D. |
| BBC21 | 4 | 906.08 | | N.D. |
| BBC27 | 6 | 934.14 | | N.D. |

TABLE 3
Characterization of Library 3 (Formula IV)
Library 3

Characterization

| | | | Mw + H | |
|---|---|---|---|---|
| Peptide | | Mw − Cal | Found | Purity |
| name | n | (gr/mol) | (gr/mol) | (%) |
| BBC16 | 2 | 883.99 | 885.14 | 96.2 |
| BBC18 | 3 | 898.02 | | N.D. |
| BBC20 | 4 | 912.05 | | N.D. |
| BBC26 | 6 | 940.10 | | N.D. |

N.D.—not determined

TABLE 4
IC₅₀ values of BBC peptides

| Peptide | IC₅₀ nM |
|---|---|
| BBC1 | 90 |
| BBC6 | 110 |
| BBC8 | 60 |
| BBC7 | 100 |
| BBC9 | 100 |
| BBC10 | 120 |
| BBC12 | 100 |
| BBC13 | 100 |
| BBC15 | 60 |
| BBC16 | 100 |
| BBC17 | 90 |

### Example 9

### In-Vivo Study to Assess the Effect of Orally Administered BBC-1 on Food Consumption in Normal Mice

ICR:Hsd (CD-1) male mice, 7-8 weeks old, were raised in separate cages and maintained at 23±1° C. on a 12-hr light, 12-hr dark cycle (0700-1900 hr light). Mice were allowed ad libitum access to water and standard chow pellets. Upon arrival mice were allowed to acclimate for 1 week. Following fasting for 16 hours, the animals (n=8) were subjected to a single oral gavage (PO, 5 ml/kg) of BBC1 (100 μg/ml, 1000 μg/ml) or vehicle (water). Immediately after administration, fixed food doses were added and re-weighed after 1, 2, 3, 4, 5, 8 and 24 hours.

The mice did not show any special clinical signs post administration of the test item during the following 24 hours. As demonstrated in FIG. 7, BBC-1 reduced food consumption in mice over a period of 24 hr by 40% when administrated orally.

These results indicate that by utilizing backbone cyclization it is possible to synthesize bioactive peptides that are stable in the intestinal milieu and cross the intestinal wall, thus, possessing potentially good oral bioavailability while maintaining their pharmacological activity.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It should be understood that the detailed description and specific examples, while indicating preferred embodiments of the invention, are given by way of illustration only, since various changes and modifications within the spirit and scope of the invention will become apparent to those skilled in the art from this detailed description.

1.-50. (canceled)

51. A backbone cyclized αMSH analog comprising a peptide sequence of four to twelve amino acids that incorporates at least one building unit, said building unit, containing one nitrogen atom of the peptide backbone connected to a bridging group comprising a disulfide, amide, thioether, thioester, imine, ether, or alkene bridge, wherein at least one building unit is connected via the bridging group to a moiety selected from the group consisting of a second building unit, a side chain of an amino acid residue of the peptide sequence, and a N-terminal amino acid residue, to form a cyclic structure.

52. The αMSH analog of claim 51 wherein the bridging group is a chemical linker having the general Formula (VII):
-Z-(CH₂)ₘ-M-(CH₂)ₙ- Formula (VII)
wherein m and n are each independently an integer for 1 to 8: M is selected from the group consisting of a disulfide, amide, thioether, thioester, imine, ether, or alkene bridge and Z is absent or is the residue of a molecule comprising two carboxylic groups.

53. The backbone cyclized αMSH analog of claim 51 having the general Formula (I) (SEQ ID NO: 2): 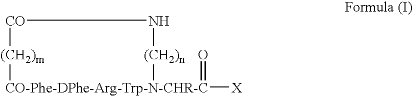
wherein R is the side chain of an amino acid, X is OH, NH₂ or an ester, m denotes an integer from 1 to 8 and n denotes an integer from 1 to 8.

54. The backbone cyclized αMSH analog of claim 53 wherein m denotes an integer from 2 to 5 and n denotes an integer from 2 to 6.

55. The backbone cyclized αMSH analog of claim 53 having the general formula (II) (SEQ ID NO: 3): 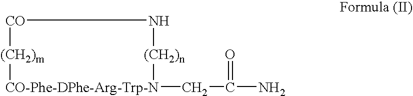
wherein m denotes an integer from 1 to 8 and n denotes an integer from 1 to 8.

56. The backbone cyclized αMSH analog of claim 55 wherein m denotes an integer from 2 to 5 and n denotes an integer from 2 to 6.

57. The backbone cyclized αMSH analog of claim 56 wherein said analog is selected from the group consisting of:
a peptide according to Formula II wherein n=2, m=2;
a peptide according to Formula II wherein n=3, m=3;
a peptide according to Formula II wherein n=3, m=2;
a peptide according to Formula II wherein n=3, m=5; and
a peptide according to Formula II wherein n=2, m=4.

58. The backbone cyclized αMSH analog of claim 51 having the general formula (III): 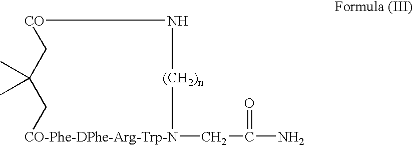
wherein n denotes an integer from 1 to 8.

59. The backbone cyclized αMSH analog of claim 58 wherein n denotes an integer from 2 to 6.

60. The backbone cyclized αMSH analog of claim 58 wherein n denotes an integer selected from 2, 3, 4, and 6.

61. The backbone cyclized αMSH analog of claim 51 having the general formula (IV): 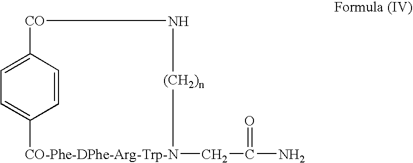
wherein n denotes an integer from 1 to 8.

62. The backbone cyclized αMSH analog of claim 61 wherein n denotes an integer from 2 to 6.

63. The backbone cyclized αMSH analog of claim 61 wherein n denotes an integer selected from 2, 3, 4, and 6.

64. A pharmaceutical composition comprising as an active ingredient a backbone cyclized αMSH analog according to claim 51, further comprising a pharmaceutically acceptable carrier.

65. The pharmaceutical composition of claim 64 which is formulated for oral administration.

66. A method for treatment or prophylaxis of diseases or disorders which are associated with melanocortin-4-receptor activity, comprises administering to a subject in need thereof a therapeutically effective amount of a pharmaceutical composition comprising as an active ingredient a backbone cyclized αMSH analog according to claim 51.

67. The method of claim 66 wherein the disorders are metabolic disorders.

68. The method of claim 67 wherein the metabolic disorder is obesity or diabetes.

69. The method of claim 67 wherein the metabolic disorder is diabetes type II.

70. The method of claim 66 wherein the pharmaceutical composition is formulated for oral administration.

71. The method of claim 66 wherein the amount of the active ingredient is in the range of from about 10 to 1000 μg/kg.
